(19) United States
(12) Patent Application Publication
(10) Pub. No.: US 2022/0130505 A1
(43) Pub. Date: Apr. 28, 2022

(54) Method, System, and Computer Program Product for Pharmacy Substitutions

(71) Applicant: Axixe LLC, Wexford, PA (US)

(72) Inventors: Eric Brannon Molitor, Sewickley, PA (US); Thomas William Light, Rostraver Twp, PA (US); Sean Charles O'Brien, Wexford, PA (US)

(21) Appl. No.: 17/510,990

(22) Filed: Oct. 26, 2021

Related U.S. Application Data
(60) Provisional application No. 63/106,214, filed on Oct. 27, 2020.

(51) Int. Cl. G16H 20/10; G06N 20/00; G16H 50/20

(52) U.S. Cl. CPC G16H 20/10; G16H 50/20; G06N 20/00

(57) ABSTRACT

Methods, systems, and computer program products for pharmacy substitutions obtain claims data associated with a claim for a prescription associated with a patient; determine, based on the claims data, a universal identifier for the prescription; obtain drug diagnosis data associated with known diagnoses for universal identifiers associated with prescriptions; determine, based on the universal identifier and the drug diagnosis data, a likely diagnosis associated with the prescription; determine, based on the claims data, a cost associated with the likely diagnosis; determine, for the likely diagnosis, using a machine learning model trained based on a training dataset, a potential alternative prescription to the prescription; update, based on user input, the training dataset to include the likely diagnosis associated with the alternative prescription; and train the machine learning model based on the updated training dataset.

### CROSS-REFERENCE TO RELATED APPLICATION

The present application claims priority to U.S. Provisional Application Ser. No. 63/106,214, entitled “Method, System, and Computer Program Product for Pharmacy Substitutions”, filed Oct. 27, 2020, the entire disclosure of which is incorporated by reference in its entirety.

### BACKGROUND

### 1. Field

This disclosure relates to pharmacy substitutions and, in some non-limiting embodiments or aspects, to methods, systems, and computer program products for training and updating a machine learning process for determining an alternative prescription to a prescription for a diagnosis.

### 2. Technical Considerations

Pharmacists may recommend substituting one or more prescriptions for one or more other prescriptions prescribed for a condition/disease of a patient to realize a cost savings provided by the one or more prescriptions over the one or more other prescriptions.

### SUMMARY

Accordingly, provided are improved systems, devices, products, apparatus, and/or methods for pharmacy substitutions.

According to some non-limiting embodiments or aspects, provided is a computer-implemented method including: obtaining claims data associated with at least one claim for at least one prescription associated with at least one patient; determining, based on the claims data, at least one universal identifier for the at least one prescription associated with the at least one patient; obtaining drug diagnosis data associated with one or more known diagnoses for one or more universal identifiers associated with one or more prescriptions; determining, based on the at least one universal identifier and the drug diagnosis data, at least one likely diagnosis associated with the at least one prescription for the at least one patient; determining, based on the claims data, at least one cost associated with the at least one likely diagnosis associated with the at least one prescription; determining, for the at least one likely diagnosis, using a machine learning model trained based on a training dataset, at least one potential alternative prescription to the at least one prescription associated with the at least one likely diagnosis; providing, to at least one user, savings information associated with the at least one potential alternative prescription to the at least one prescription associated with the at least one likely diagnosis; receiving, from the at least one user, user input associated with the at least one potential alternative prescription to the at least one prescription associated with the at least one likely diagnosis; updating, based on the user input, the training dataset to include the at least one likely diagnosis associated with the at least one potential alternative prescription as at least one trained diagnosis; and training the machine learning model based on the updated training dataset.

In some non-limiting embodiments or aspects, the method further includes: determining whether the at least one likely diagnosis matches one or more confirmed diagnoses in a confirmed dataset; in response to determining that the at least one likely diagnosis matches one or more confirmed diagnoses in a confirmed dataset, one of: (i) updating the training dataset to include the at least one likely diagnosis associated with the at least one prescription as one or more trained diagnosis and (ii) updating the trained dataset by adjusting a weighting associated with at least one existing trained diagnosis in the trained dataset.

In some non-limiting embodiments or aspects, the method further includes: determining, based on the at least one likely diagnosis and the confirmed data set, a severity level associated with the at least one likely diagnosis, wherein severity level is input to the at least one machine learning model to determine the at least one potential alternative prescription to the at least one prescription associated with the at least one likely diagnosis.

In some non-limiting embodiments or aspects, the at least one potential alternative prescription is associated with a same severity level for the at least one likely diagnosis as the at least one prescription.

In some non-limiting embodiments or aspects, the method further includes: determining, based on the at least one likely diagnosis and the training dataset, at least one probability associated with the at least one likely diagnosis, wherein the at least one probability is input to the at least one machine learning model to determine the at least one potential alternative prescription to the at least one prescription associated with the at least one likely diagnosis.

In some non-limiting embodiments or aspects, the at least one potential alternative prescription includes at least one of the following: a different drug than a drug associated with the at least one prescription, a different dosage than a dosage associated with the at least one prescription, an indication to discontinue use of the drug associated with the at least one prescription, a different formulation of the same drug associated with the at least one prescription, a different packaging of the same drug associated with the at least one prescription, or any combination thereof.

In some non-limiting embodiments or aspects, the at least one cost is further determined based on a current cost associated with the at least one prescription and a future cost associated with the at least one prescription, wherein the future cost is different than the current cost.

According to some non-limiting embodiments or aspects, provided is a system including: one or more processors programmed and/or configured to: obtain claims data associated with at least one claim for at least one prescription associated with at least one patient; determine, based on the claims data, at least one universal identifier for the at least one prescription associated with the at least one patient; obtain drug diagnosis data associated with one or more known diagnoses for one or more universal identifiers associated with one or more prescriptions; determine, based on the at least one universal identifier and the drug diagnosis data, at least one likely diagnosis associated with the at least one prescription for the at least one patient; determine, based on the claims data, at least one cost associated with the at least one likely diagnosis associated with the at least one prescription; determine, for the at least one likely diagnosis, using a machine learning model trained based on a training dataset, at least one potential alternative prescription to the at least one prescription associated with the at least one likely diagnosis; provide, to at least one user, savings information associated with the at least one potential alternative prescription to the at least one prescription associated with the at least one likely diagnosis; receive, from the at least one user, user input associated with the at least one potential alternative prescription to the at least one prescription associated with the at least one likely diagnosis; update, based on the user input, the training dataset to include the at least one likely diagnosis associated with the at least one potential alternative prescription as at least one trained diagnosis; and train the machine learning model based on the updated training dataset.

In some non-limiting embodiments or aspects, the one or more processors are further programmed and/or configured to: determine whether the at least one likely diagnosis matches one or more confirmed diagnoses in a confirmed dataset; in response to determining that the at least one likely diagnosis matches one or more confirmed diagnoses in a confirmed dataset, one of: (i) update the trained dataset to include the at least one likely diagnosis associated with the at least one prescription as one or more trained diagnosis and (ii) update the trained dataset by adjusting a weighting associated with at least one existing trained diagnosis in the trained dataset.

In some non-limiting embodiments or aspects, the one or more processors are further programmed and/or configured to: determine, based on the at least one likely diagnosis and the confirmed data set, a severity level associated with the at least one likely diagnosis, wherein severity level is input to the at least one machine learning model to determine the at least one potential alternative prescription to the at least one prescription associated with the at least one likely diagnosis.

In some non-limiting embodiments or aspects, the at least one potential alternative prescription is associated with a same severity level for the at least one likely diagnosis as the at least one prescription.

In some non-limiting embodiments or aspects, the one or more processors are further programmed and/or configured to: determine, based on the at least one likely diagnosis and the training dataset, at least one probability associated with the at least one likely diagnosis, wherein the at least one probability is input to the at least one machine learning model to determine the at least one potential alternative prescription to the at least one prescription associated with the at least one likely diagnosis.

In some non-limiting embodiments or aspects, the at least one potential alternative prescription includes at least one of the following: a different drug than a drug associated with the at least one prescription, a different dosage than a dosage associated with the at least one prescription, an indication to discontinue use of the drug associated with the at least one prescription, a different formulation of the same drug associated with the at least one prescription, a different packaging of the same drug associated with the at least one prescription, or any combination thereof.

In some non-limiting embodiments or aspects, the at least one cost is further determined based on a current cost associated with the at least one prescription and a future cost associated with the at least one prescription, wherein the future cost is different than the current cost.

According to some non-limiting embodiments or aspects, provided is a computer program product comprising at least one non-transitory computer-readable medium including program instructions that, when executed by at least one processor, cause the at least one processor to: obtain claims data associated with at least one claim for at least one prescription associated with at least one patient; determine, based on the claims data, at least one universal identifier for the at least one prescription associated with the at least one patient; obtain drug diagnosis data associated with one or more known diagnoses for one or more universal identifiers associated with one or more prescriptions; determine, based on the at least one universal identifier and the drug diagnosis data, at least one likely diagnosis associated with the at least one prescription for the at least one patient; determine, based on the claims data, at least one cost associated with the at least one likely diagnosis associated with the at least one prescription; determine, for the at least one likely diagnosis, using a machine learning model trained based on a training dataset, at least one potential alternative prescription to the at least one prescription associated with the at least one likely diagnosis; provide, to at least one user, savings information associated with the at least one potential alternative prescription to the at least one prescription associated with the at least one likely diagnosis; receive, from the at least one user, user input associated with the at least one potential alternative prescription to the at least one prescription associated with the at least one likely diagnosis; update, based on the user input, the training dataset to include the at least one likely diagnosis associated with the at least one potential alternative prescription as at least one trained diagnosis; and train the machine learning model based on the updated training dataset.

In some non-limiting embodiments or aspects, the instructions, when executed by the at least one processor, further cause the at least one processor to: determine whether the at least one likely diagnosis matches one or more confirmed diagnoses in a confirmed dataset; and in response to determining that the at least one likely diagnosis matches one or more confirmed diagnoses in a confirmed dataset, one of: (i) update the trained dataset to include the at least one likely diagnosis associated with the at least one prescription as one or more trained diagnosis and (ii) update the trained dataset by adjusting a weighting associated with at least one existing trained diagnosis in the trained dataset.

In some non-limiting embodiments or aspects, the instructions, when executed by the at least one processor, further cause the at least one processor to: determine, based on the at least one likely diagnosis and the confirmed data set, a severity level associated with the at least one likely diagnosis, wherein severity level is input to the at least one machine learning model to determine the at least one potential alternative prescription to the at least one prescription associated with the at least one likely diagnosis.

In some non-limiting embodiments or aspects, the at least one potential alternative prescription is associated with a same severity level for the at least one likely diagnosis as the at least one prescription.

In some non-limiting embodiments or aspects, the instructions, when executed by the at least one processor, further cause the at least one processor to: determine, based on the at least one likely diagnosis and the training dataset, at least one probability associated with the at least one likely diagnosis, wherein the at least one probability is input to the at least one machine learning model to determine the at least one potential alternative prescription to the at least one prescription associated with the at least one likely diagnosis.

In some non-limiting embodiments or aspects, the at least one potential alternative prescription includes at least one of the following: a different drug than a drug associated with the at least one prescription, a different dosage than a dosage associated with the at least one prescription, an indication to discontinue use of the drug associated with the at least one prescription, a different formulation of the same drug associated with the at least one prescription, a different packaging of the same drug associated with the at least one prescription, or any combination thereof.

In some non-limiting embodiments or aspects, the at least one cost is further determined based on a current cost associated with the at least one prescription and a future cost associated with the at least one prescription, wherein the future cost is different than the current cost.

Further non-limiting embodiments or aspects are set forth in the following numbered clauses:

Clause 1. A computer-implemented method comprising: obtaining claims data associated with at least one claim for at least one prescription associated with at least one patient; determining, based on the claims data, at least one universal identifier for the at least one prescription associated with the at least one patient; obtaining drug diagnosis data associated with one or more known diagnoses for one or more universal identifiers associated with one or more prescriptions; determining, based on the at least one universal identifier and the drug diagnosis data, at least one likely diagnosis associated with the at least one prescription for the at least one patient; determining, based on the claims data, at least one cost associated with the at least one likely diagnosis associated with the at least one prescription; determining, for the at least one likely diagnosis, using a machine learning model trained based on a training dataset, at least one potential alternative prescription to the at least one prescription associated with the at least one likely diagnosis; providing, to at least one user, savings information associated with the at least one potential alternative prescription to the at least one prescription associated with the at least one likely diagnosis; receiving, from the at least one user, user input associated with the at least one potential alternative prescription to the at least one prescription associated with the at least one likely diagnosis; updating, based on the user input, the training dataset to include the at least one likely diagnosis associated with the at least one potential alternative prescription as at least one trained diagnosis; and training the machine learning model based on the updated training dataset.

Clause 2. The computer-implemented method of clause 1, further comprising: determining whether the at least one likely diagnosis matches one or more confirmed diagnoses in a confirmed dataset; in response to determining that the at least one likely diagnosis matches one or more confirmed diagnoses in a confirmed dataset, one of: (i) updating the training dataset to include the at least one likely diagnosis associated with the at least one prescription as one or more trained diagnosis and (ii) updating the trained dataset by adjusting a weighting associated with at least one existing trained diagnosis in the trained dataset.

Clause 3. The computer-implemented method of any of clauses 1 and 2, further comprising: determining, based on the at least one likely diagnosis and the confirmed data set, a severity level associated with the at least one likely diagnosis, wherein severity level is input to the at least one machine learning model to determine the at least one potential alternative prescription to the at least one prescription associated with the at least one likely diagnosis.

Clause 4. The computer-implemented method of any of clauses 1-3, wherein the at least one potential alternative prescription is associated with a same severity level for the at least one likely diagnosis as the at least one prescription.

Clause 5. The computer-implemented method of any of clauses 1-4, further comprising: determining, based on the at least one likely diagnosis and the training dataset, at least one probability associated with the at least one likely diagnosis, wherein the at least one probability is input to the at least one machine learning model to determine the at least one potential alternative prescription to the at least one prescription associated with the at least one likely diagnosis.

Clause 6. The computer-implemented method of any of clauses 1-5, wherein the at least one potential alternative prescription includes at least one of the following: a different drug than a drug associated with the at least one prescription, a different dosage than a dosage associated with the at least one prescription, an indication to discontinue use of the drug associated with the at least one prescription, a different formulation of the same drug associated with the at least one prescription, a different packaging of the same drug associated with the at least one prescription, or any combination thereof.

Clause 7. The computer-implemented method of any of clauses 1-6, wherein the at least one cost is further determined based on a current cost associated with the at least one prescription and a future cost associated with the at least one prescription, wherein the future cost is different than the current cost.

Clause 8. A system comprising: one or more processors programmed and/or configured to: obtain claims data associated with at least one claim for at least one prescription associated with at least one patient; determine, based on the claims data, at least one universal identifier for the at least one prescription associated with the at least one patient; obtain drug diagnosis data associated with one or more known diagnoses for one or more universal identifiers associated with one or more prescriptions; determine, based on the at least one universal identifier and the drug diagnosis data, at least one likely diagnosis associated with the at least one prescription for the at least one patient; determine, based on the claims data, at least one cost associated with the at least one likely diagnosis associated with the at least one prescription; determine, for the at least one likely diagnosis, using a machine learning model trained based on a training dataset, at least one potential alternative prescription to the at least one prescription associated with the at least one likely diagnosis; provide, to at least one user, savings information associated with the at least one potential alternative prescription to the at least one prescription associated with the at least one likely diagnosis; receive, from the at least one user, user input associated with the at least one potential alternative prescription to the at least one prescription associated with the at least one likely diagnosis; update, based on the user input, the training dataset to include the at least one likely diagnosis associated with the at least one potential alternative prescription as at least one trained diagnosis; and train the machine learning model based on the updated training dataset.

Clause 9. The system of clause 8, wherein the one or more processors are further programmed and/or configured to: determine whether the at least one likely diagnosis matches one or more confirmed diagnoses in a confirmed dataset; in response to determining that the at least one likely diagnosis matches one or more confirmed diagnoses in a confirmed dataset, one of: (i) update the trained dataset to include the at least one likely diagnosis associated with the at least one prescription as one or more trained diagnosis and (ii) update the trained dataset by adjusting a weighting associated with at least one existing trained diagnosis in the trained dataset.

Clause 10. The system of any of clauses 8 and 9, wherein the one or more processors are further programmed and/or configured to: determine, based on the at least one likely diagnosis and the confirmed data set, a severity level associated with the at least one likely diagnosis, wherein severity level is input to the at least one machine learning model to determine the at least one potential alternative prescription to the at least one prescription associated with the at least one likely diagnosis.

Clause 11. The system of any of clauses 8-10, wherein the at least one potential alternative prescription is associated with a same severity level for the at least one likely diagnosis as the at least one prescription.

Clause 12. The system of any of clauses 8-11, wherein the one or more processors are further programmed and/or configured to: determine, based on the at least one likely diagnosis and the training dataset, at least one probability associated with the at least one likely diagnosis, wherein the at least one probability is input to the at least one machine learning model to determine the at least one potential alternative prescription to the at least one prescription associated with the at least one likely diagnosis.

Clause 13. The system of any of clauses 8-12, wherein the at least one potential alternative prescription includes at least one of the following: a different drug than a drug associated with the at least one prescription, a different dosage than a dosage associated with the at least one prescription, an indication to discontinue use of the drug associated with the at least one prescription, a different formulation of the same drug associated with the at least one prescription, a different packaging of the same drug associated with the at least one prescription, or any combination thereof.

Clause 14. The system of any of clauses 8-13, wherein the at least one cost is further determined based on a current cost associated with the at least one prescription and a future cost associated with the at least one prescription, wherein the future cost is different than the current cost.

Clause 15. A computer program product comprising at least one non-transitory computer-readable medium including program instructions that, when executed by at least one processor, cause the at least one processor to: obtain claims data associated with at least one claim for at least one prescription associated with at least one patient; determine, based on the claims data, at least one universal identifier for the at least one prescription associated with the at least one patient; obtain drug diagnosis data associated with one or more known diagnoses for one or more universal identifiers associated with one or more prescriptions; determine, based on the at least one universal identifier and the drug diagnosis data, at least one likely diagnosis associated with the at least one prescription for the at least one patient; determine, based on the claims data, at least one cost associated with the at least one likely diagnosis associated with the at least one prescription; determine, for the at least one likely diagnosis, using a machine learning model trained based on a training dataset, at least one potential alternative prescription to the at least one prescription associated with the at least one likely diagnosis; provide, to at least one user, savings information associated with the at least one potential alternative prescription to the at least one prescription associated with the at least one likely diagnosis; receive, from the at least one user, user input associated with the at least one potential alternative prescription to the at least one prescription associated with the at least one likely diagnosis; update, based on the user input, the training dataset to include the at least one likely diagnosis associated with the at least one potential alternative prescription as at least one trained diagnosis; and train the machine learning model based on the updated training dataset.

Clause 16. The computer program product of clause 15, wherein the instructions, when executed by the at least one processor, further cause the at least one processor to: determine whether the at least one likely diagnosis matches one or more confirmed diagnoses in a confirmed dataset; in response to determining that the at least one likely diagnosis matches one or more confirmed diagnoses in a confirmed dataset, one of: (i) update the trained dataset to include the at least one likely diagnosis associated with the at least one prescription as one or more trained diagnosis and (ii) update the trained dataset by adjusting a weighting associated with at least one existing trained diagnosis in the trained dataset.

Clause 17. The computer program product of any of clauses 15 and 16, wherein the instructions, when executed by the at least one processor, further cause the at least one processor to: determine, based on the at least one likely diagnosis and the confirmed data set, a severity level associated with the at least one likely diagnosis, wherein severity level is input to the at least one machine learning model to determine the at least one potential alternative prescription to the at least one prescription associated with the at least one likely diagnosis.

Clause 18. The computer program product of any of clauses 15-17, wherein the at least one potential alternative prescription is associated with a same severity level for the at least one likely diagnosis as the at least one prescription.

Clause 19. The computer program product of any of clauses 15-18, wherein the instructions, when executed by the at least one processor, further cause the at least one processor to: determine, based on the at least one likely diagnosis and the training dataset, at least one probability associated with the at least one likely diagnosis, wherein the at least one probability is input to the at least one machine learning model to determine the at least one potential alternative prescription to the at least one prescription associated with the at least one likely diagnosis.

Clause 20. The computer program product of any of clauses 15-19, wherein the at least one potential alternative prescription includes at least one of the following: a different drug than a drug associated with the at least one prescription, a different dosage than a dosage associated with the at least one prescription, an indication to discontinue use of the drug associated with the at least one prescription, a different formulation of the same drug associated with the at least one prescription, a different packaging of the same drug associated with the at least one prescription, or any combination thereof.

Clause 21. The computer program product of any of clauses 15-20, wherein the at least one cost is further determined based on a current cost associated with the at least one prescription and a future cost associated with the at least one prescription, wherein the future cost is different than the current cost.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of limits. As used in the specification and the claims, the singular form of “a,” “an,” and “the” include plural referents unless the context clearly dictates otherwise.

### BRIEF DESCRIPTION OF THE DRAWINGS AND APPENDICES

Additional advantages and details are explained in greater detail below with reference to the exemplary embodiments that are illustrated in the accompanying schematic figures, in which:

FIG. 1 is a diagram of non-limiting embodiments or aspects of an environment in which systems, devices, products, apparatus, and/or methods, described herein, may be implemented;

FIG. 2 is a diagram of non-limiting embodiments or aspects of components of one or more devices and/or one or more systems of FIG. 1;

FIGS. 3A and 3B are a flowchart of non-limiting embodiments or aspects of a process for pharmacy substitutions;

FIG. 4 is a flowchart of non-limiting embodiments or aspects of a process for pharmacy substitutions;

FIG. 5 is a flowchart of non-limiting embodiments or aspects of a process for pharmacy substitutions;

FIG. 6 is a flowchart of non-limiting embodiments or aspects of a process for pharmacy substitutions; and

FIG. 7 illustrates example potential rules that may be discovered by a process for pharmacy substitutions.

### DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the term “communication” may refer to the reception, receipt, transmission, transfer, provision, and/or the like of data (e.g., information, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit processes information received from the first unit and communicates the processed information to the second unit.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

As used herein, the term “mobile device” may refer to one or more portable electronic devices configured to communicate with one or more networks. As an example, a mobile device may include a cellular phone (e.g., a smartphone or standard cellular phone), a portable computer (e.g., a tablet computer, a laptop computer, etc.), a wearable device (e.g., a watch, pair of glasses, lens, clothing, and/or the like), a personal digital assistant (PDA), and/or other like devices. The terms “client device” and “user device,” as used herein, refer to any electronic device that is configured to communicate with one or more servers or remote devices and/or systems. A client device or user device may include a mobile device, a network-enabled appliance (e.g., a network-enabled television, refrigerator, thermostat, and/or the like), a computer, a POS system, and/or any other device or system capable of communicating with a network.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a PDA, and/or other like devices. A computing device may also be a desktop computer or other form of non-mobile computer.

As used herein, the term “server” and/or “processor” may refer to or include one or more computing devices that are operated by or facilitate communication and processing for multiple parties in a network environment, such as the Internet, although it will be appreciated that communication may be facilitated over one or more public or private network environments and that various other arrangements are possible. Further, multiple computing devices (e.g., servers, POS devices, mobile devices, etc.) directly or indirectly communicating in the network environment may constitute a “system.” Reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

As used herein, the term “application programming interface” (API) may refer to computer code that allows communication between different systems or (hardware and/or software) components of systems. For example, an API may include function calls, functions, subroutines, communication protocols, fields, and/or the like usable and/or accessible by other systems or other (hardware and/or software) components of systems.

As used herein, the term “user interface” or “graphical user interface” refers to a generated display, such as one or more graphical user interfaces (GUIs) with which a user may interact, either directly or indirectly (e.g., through a keyboard, mouse, touchscreen, etc.).

Non-limiting embodiments or aspects of the present disclosure may compare patient medical and drug claims to those of other patients for determination of likely diagnosis, drug to diagnosis equivalencies, and/or diagnosis severity and use that determination to identify drug and drug combinations that reduce or minimize a cost of care by suggesting clinically equivalent changes to drug prescriptions. For example, non-limiting embodiments or aspects of the present disclosure may learn and understand nuance(s) of clinical equivalency in prescriptions. As an example, for a patient taking a 10 mg tablet of a drug that costs $X, where that same drug is now available in a 25 mg dosage also costing $X, a pharmacist may split the 25 mg tablet in half and save 50% by knowing half of 25 mg is clinically equivalent to 10 mg. In such an example, non-limiting embodiments or aspects of the present disclosure may automatically learn to recognize that 10 mg is clinically equal to 12.5 mg (e.g., a learned/trained behavior that X can equal Y, for example, when certain other criteria are met, etc.)

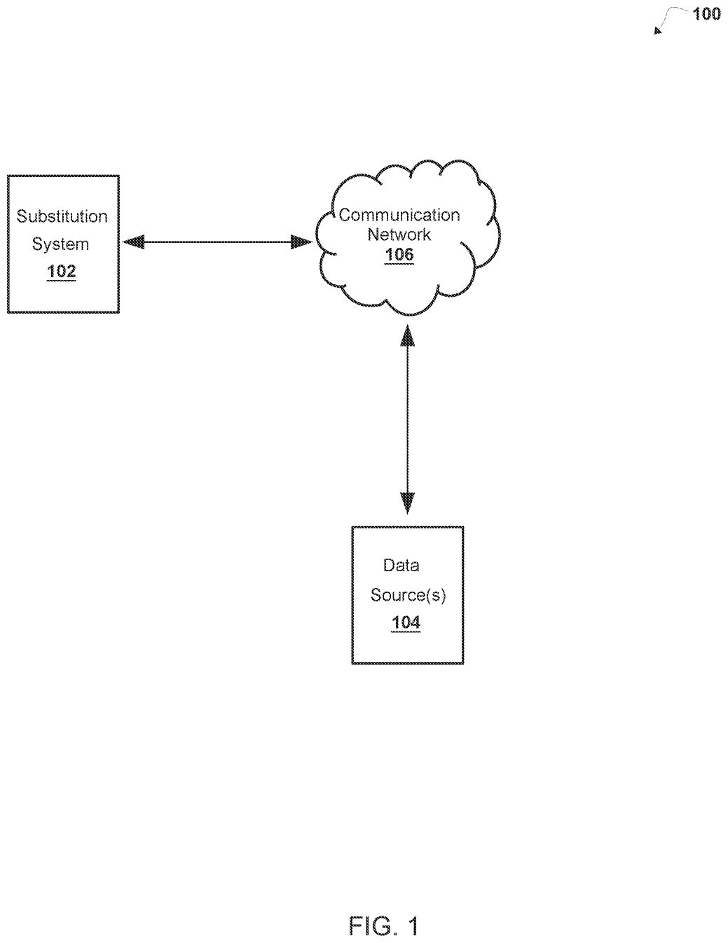

Referring now to FIG. 1, FIG. 1 is a diagram of an example environment 100 in which devices, systems, methods, and/or products described herein, may be implemented. As shown in FIG. 1, environment 100 includes transaction substitution system 102, one or more data sources 104, and/or communication network 106. Substitution system 102 and the one or more data sources may interconnect (e.g., establish a connection to communicate, etc.) via wired connections, wireless connections, or a combination of wired and wireless connections.

Substitution system 102 may include one or more devices capable of receiving information and/or data from the one or more data sources 104 via communication network 106 and/or communicating information and/or data to the one or more data sources 104 via communication network 106. For example, substitution system 102 may include a computing device, such as a server, a group of servers, and/or other like devices.

The one or more data sources 104 may include one or more devices capable of receiving information and/or data from substitution system 102 via communication network 106 and/or communicating information and/or data to substitution system 102 via communication network 106. For example, the one or more data sources may include a computing device, such as a server, a group of servers, and/or other like devices. In some non-limiting embodiments or aspects, the one or more data sources 104 may include one or more databases.

Communication network 106 may include one or more wired and/or wireless networks. For example, communication network 106 may include a cellular network (e.g., a long-term evolution (LTE) network, a third generation (3G) network, a fourth generation (4G) network, a fifth generation (5G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of these or other types of networks.

The number and arrangement of devices and systems shown in FIG. 1 is provided as an example. There may be additional devices and/or systems, fewer devices and/or systems, different devices and/or systems, or differently arranged devices and/or systems than those shown in FIG. 1. Furthermore, two or more devices and/or systems shown in FIG. 1 may be implemented within a single device and/or system, or a single device and/or system shown in FIG. 1 may be implemented as multiple, distributed devices and/or systems. Additionally, or alternatively, a set of devices and/or systems (e.g., one or more devices or systems) of environment 100 may perform one or more functions described as being performed by another set of devices and/or systems of environment 100.

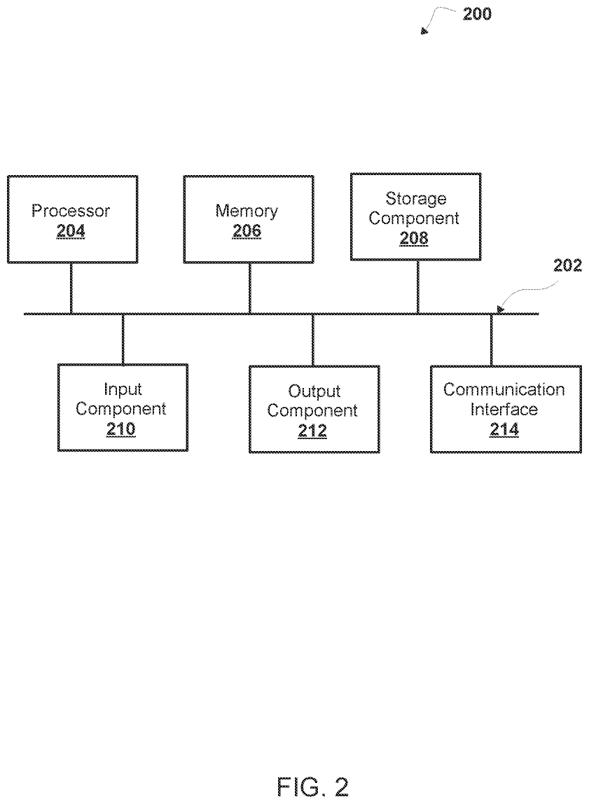

Referring now to FIG. 2, FIG. 2 is a diagram of example components of a device 200. Device 200 may correspond to one or more devices of substitution system 102 and/or one or more devices of the one or more data sources 104. In some non-limiting embodiments or aspects, one or more devices of substitution system 102 and/or one or more devices of the one or more data sources 104 may include at least one device 200 and/or at least one component of device 200. As shown in FIG. 2, device 200 may include a bus 202, a processor 204, memory 206, a storage component 208, an input component 210, an output component 212, and a communication interface 214.

Bus 202 may include a component that permits communication among the components of device 200. In some non-limiting embodiments or aspects, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touch screen display, a keyboard, a keypad, a mouse, a button, a switch, a microphone, etc.). Additionally, or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 (e.g., a central processing unit (CPU), a graphics processing unit (GPU), etc.) executing software instructions stored by a computer-readable medium, such as memory 206 and/or storage component 208. A computer-readable medium (e.g., a non-transitory computer-readable medium) is defined herein as a non-transitory memory device. A memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally, or alternatively, hardwired circuitry may be used in place of or in combination with software instructions to perform one or more processes described herein. Thus, embodiments or aspects described herein are not limited to any specific combination of hardware circuitry and software.

Memory 206 and/or storage component 208 may include data storage or one or more data structures (e.g., a database, etc.). Device 200 may be capable of receiving information from, storing information in, communicating information to, or searching information stored in the data storage or one or more data structures in memory 206 and/or storage component 208.

The number and arrangement of components shown in FIG. 2 are provided as an example. In some non-limiting embodiments or aspects, device 200 may include additional components, fewer components, different components, or differently arranged components than those shown in FIG. 2. Additionally, or alternatively, a set of components (e.g., one or more components) of device 200 may perform one or more functions described as being performed by another set of components of device 200.

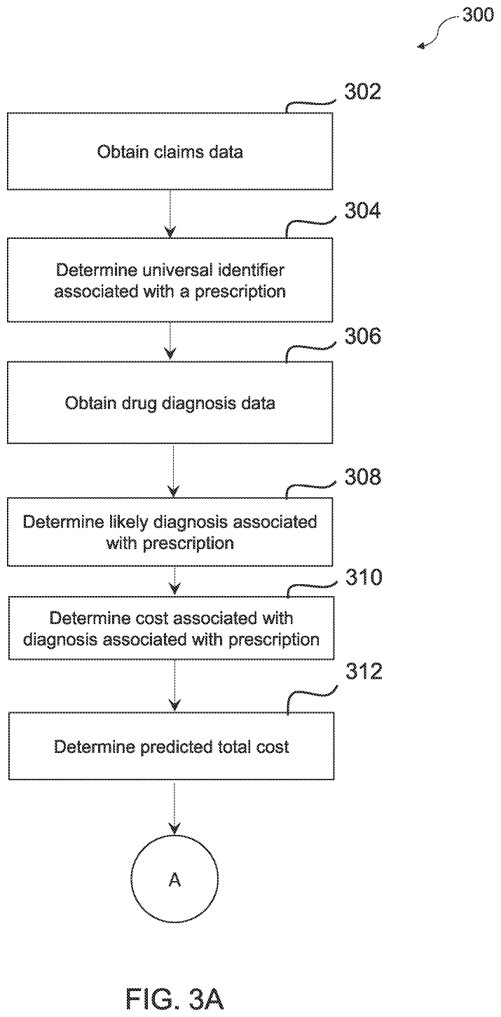

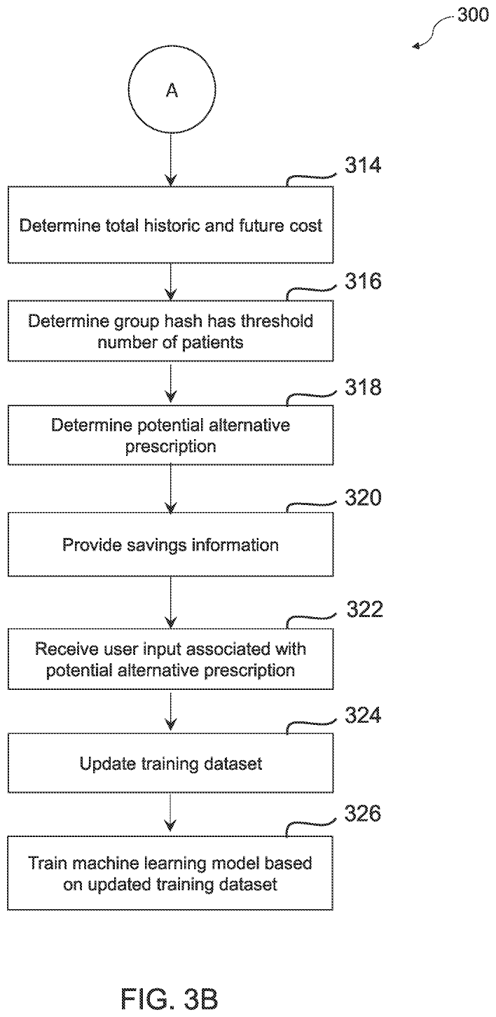

Referring now to FIGS. 3A and 3B, FIGS. 3A and 3B are a flowchart of non-limiting embodiments or aspects of a process 300 for pharmacy substitutions. In some non-limiting embodiments or aspects, one or more of the steps of process 300 may be performed (e.g., completely, partially, etc.) by substitution system 102 (e.g., one or more devices of substitution system 102, etc.). In some non-limiting embodiments or aspects, one or more of the steps of process 500 may be performed (e.g., completely, partially, etc.) by another device or a group of devices separate from or including substitution system 102, such as a user device (e.g., one or more devices of a system of a user device, etc.).

As shown in FIG. 3A, at step 302, process 300 includes obtaining claims data. For example, substitution system 102 may obtain claims data associated with at least one claim for at least one prescription associated with at least one patient. As an example, substitution system 102 may obtain claims data from the one or more data sources 104 (e.g., a claims database, an Electronic Health Record (EHR) database, etc.), the patient, and/or a medical professional.

Claims data may include at least one of the following parameters associated with a claim and/or a prescription associated with a patient: a claim identifier, a record identifier, a patient identifier, a patient name, a patient date of birth, a patient gender, a prescription data last filled, a prescription claim date, a prescription next fill date, an insurance paid amount, a patient co-pay amount, a primary care provider national provider identifier (e.g., NPI, GMC, etc.), a primary care provider name, a prescriber national provider identifier (e.g., NPI, GMC, etc.), a prescribed drug national and/or international drug code (e.g., NDC, MPID, PhPID, etc.), a prescribed drug quantity, a number of days' supply of a prescribed drug, a record identifier generation date, an insurance company and/or other payor, an insurance plan and/or other health benefit coverage, a patient insurance and/or health benefit coverage expiration date, a pharmacy location and/or name at which a prescription is filled, a national and/or international drug code (e.g., NDC, MPID, PhPID, etc.), a unique identifier providing normalized and/or standardized concept identifier names to clinical drugs (e.g., an RxNorm concept unique identifier (CUI), a Dictionary of Medicine and Device (dm+d) identifier, an Australian Medicine Terminology (AMT) identifier, a Drug Bank ID, etc.), a drug name, or any combination thereof.

In some non-limiting embodiments or aspects, claims data includes at least one of the following types of data: cost data, drug diagnosis data, patient diagnosis data, cost data, future data, or any combination thereof.

As shown in FIG. 3A, at step 304, process 300 includes determining a universal identifier associated with a prescription. For example, substitution system 102 may determine, based on the claims data, at least one universal identifier (e.g., an RxNorm identifier, an RxNorm CUI, dm+d, AMT, etc.) for the at least one prescription associated with the at least one patient. As an example, substitution system 102 may, for each patient, identify at least one active prescription associated with that patient based on the prescription next fill date(s) in the claims data associated with that patient having a date in the future, and/or substitution system 102 may determine, for that patient, by mapping a national and/or international drug code (e.g., NDC, MPID, PhPID, etc.) of a drug associated with the at least one active prescription to the unique identifier (e.g. RxNorm CUI, dm+d, AMT, etc.) associated with that drug (e.g., using a mapping provided by the National Library of Medicine, NHS Medicines Database, Drug Bank, etc.). Additionally, or alternatively, if a national or international drug code (e.g., NDC, MPID, etc.) to a unique identifier providing normalized and/or standardized concept identifier names to clinical drugs (e.g., RxNorm CUI/RxCUI, Drug Bank ID, etc.) mapping is not available, substitution system 102 may be trained (e.g., by a Pharmacist, as an automated Levenshtein distance mapping/matching algorithm on the drug name, etc.) to create equivalent mappings of drugs and/or prescriptions to a universal identifier.

As shown in FIG. 3A, at step 306, process 300 includes obtaining drug diagnosis data. For example, substitution system 102 may obtain drug diagnosis data associated with one or more known diagnoses for one or more universal identifiers associated with one or more prescriptions. As an example, substitution system 102 may obtain drug diagnosis data from the one or more data sources 104. In such an example, substitution system 102 may use a mapping (e.g., a mapping provided by the National Library of Medicine, NHS Medicines Database, Drug Bank Database, etc.) that maps unique identifiers (e.g. RxNorm CUIs) to known diagnoses (e.g., conditions, diseases, etc.) and/or international classification of disease (ICD) codes to determine the one or more known diagnoses (and/or ICD codes) for the one or more universal identifiers associated with the one or more prescriptions. Additionally, or alternatively, if a RxNorm CUI to a known diagnosis mapping is not available, substitution system 102 may be trained (e.g., by a Pharmacist, as an automated Levenshtein distance mapping/matching algorithm on the RxNorm CUI, etc.) to create equivalent mappings of universal identifiers to diagnoses.

In some non-limiting embodiments or aspects, drug diagnosis data includes patient diagnosis data. For example, substitution system 102 may obtain patient diagnosis data associated with one or more patients. As an example, substitution system 102 may obtain patient diagnosis data from the one or more data sources 104. In such an example, substitution system 102 may use a mapping that maps patient identifiers to confirmed diagnoses (and/or severity levels of the confirmed diagnoses) for the patients associated with the patient identifiers. For example, a confirmed diagnosis and/or a severity level thereof may be provided by a patient and/or a medical professional, and/or substitution system 102 may analyze medical claims and/or Electronic Health Records (EHR) associated with the patient to determine a confirmed diagnosis and/or a severity level thereof associated with the patient.

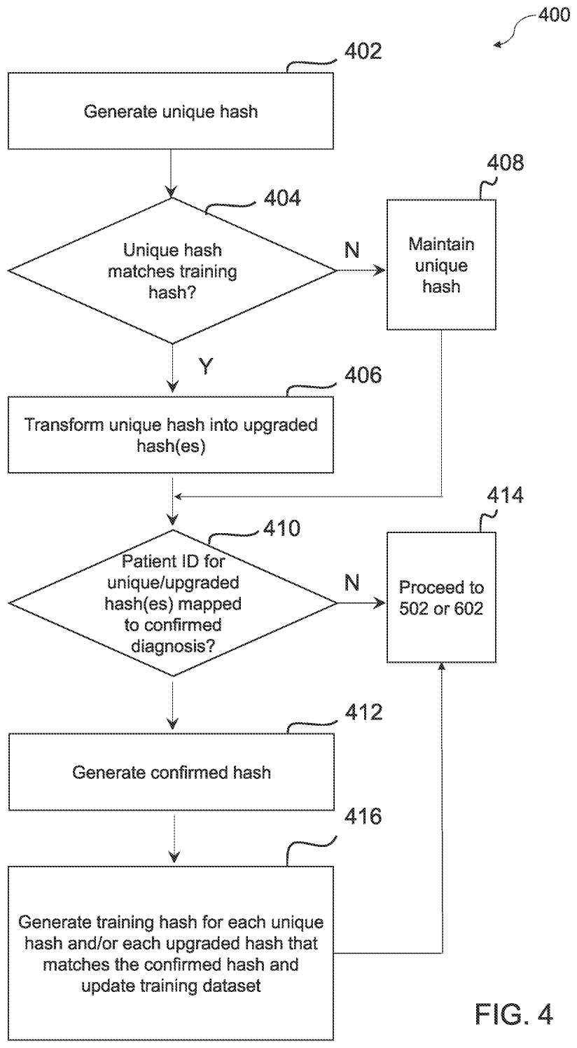

As shown in FIG. 3A, at step 308, process 300 includes determining a likely diagnosis associated with a prescription. For example, substitution system 102 may determine, based on the at least one universal identifier and the drug diagnosis data, at least one likely diagnosis associated with the at least one prescription for the at least one patient. As an example, substitution system 102 may automatically determine, using an iterative learning process and a universal identifier to known diagnosis mapping, one or more drugs that the at least one patient is prescribed for the at least one likely diagnosis. Further details regarding non-limiting embodiments or aspects of step 308 of process 300 are provided below with regard to FIGS. 4-6.

Referring now also to FIG. 4, FIG. 4 is a flowchart of non-limiting embodiments or aspects of a process 400 for pharmacy substitutions. In some non-limiting embodiments or aspects, one or more of the steps of process 400 may be performed (e.g., completely, partially, etc.) by substitution system 102 (e.g., one or more devices of substitution system 102, etc.). In some non-limiting embodiments or aspects, one or more of the steps of process 500 may be performed (e.g., completely, partially, etc.) by another device or a group of devices separate from or including substitution system 102, such as a user device (e.g., one or more devices of a system of a user device, etc.).

As shown in FIG. 4, at step 402, process 400 includes generating a unique hash associated with a patient. For example, substitution system 102 may generate a unique hash LikelyHASH associated with a patient by combining a patient identifier associated with a universal identifier (e.g., as generated at step 304, etc.) with a diagnosis associated with the same universal identifier (e.g., the same RxNorm CUI, etc.). As an example, the unique hash LikelyHASH may include an ascending list of universal identifiers (e.g., CUIs, etc.) for each likely diagnosis (e.g., diabetes, ketoacidosis, etc.) for each patient.

A patient may be associated with a plurality of unique hashes LikelyHashes if the patient is associated with multiple diagnoses. For example, for two example patients

Patient 1 and Patient 2, the following example unique hashes may be generated: Patient 1: LikelyHASH(CUI1,CUI2,CUI3) equals likely Diabetes for Patient 1, Patient 2: LikelyHASH(CU1,CUI2,CUI5) equals likely Diabetes for Patient 2, and Patient 1: LIkeIyHASH(CUI4,CUI5) equals likely Ketoacidosis for Patient 1. As an example, a unique hash LikelyHASH may include any number of CUls for a particular diagnosis, and/or CUls may overlap for different diagnoses. For example, the example unique hash LikelyHASH(CUI1,CUI2,CUI3) may also equal a likely Ketoacidosis diagnosis for example Patient 1.

As shown in FIG. 4, at step 404, process 400 includes determining whether a unique hash matches a training hash. For example, substitution system 102 may determine whether the unique hash LikelyHASH matches a TrainingHASH stored in a training dataset. As an example, substitution system 102 may compare each unique hash LikelyHASH to TrainingHASHes previously stored in a training dataset.

As shown in FIG. 4, at step 406, process 400 includes, in response to determining that a unique hash matches a training hash, transforming the unique hash into one or more upgraded hashes. For example, substitution system 102 may, in response to determining that the unique hash LikelyHASH matches one or more TrainingHASHes stored in the training dataset, transform the unique hash LikelyHASH into one or more upgraded hash(es) UpgradedHASH(es). As an example, for each match that the unique hash LikelyHASH has in the training dataset, an upgraded hash UpgradedHASH may be generated such that a single unique hash LikelyHASH may generate multiple UpgradedHASHes.

As shown in FIG. 4, at step 408, process 400 includes, in response to determining that a unique hash does not match a training hash, maintaining the unique hash. For example, substitution system 102 may, in response to determining that the unique hash LikelyHASH does not match any TrainingHASH stored in the training dataset, maintain the unique hash LikelyHASH (e.g., not transform the unique hash LikelyHASH, etc.).

As shown in FIG. 4, at step 410, process 400 includes determining whether a patient identifier associated with a unique hash (and/or an upgraded hash) is mapped to a confirmed diagnosis. For example, substitution system 102 may determine whether a patient identifier associated with the unique hash identifier LikelyHASH (and/or the upgraded hash(es) UpgradedHASH(es)) is mapped to a confirmed diagnosis. As an example, substitution system 102 may use a mapping that maps patient identifiers to confirmed diagnoses (and/or severity levels of the confirmed diagnoses) for the patients associated with the patient identifiers. For example, a confirmed diagnosis and/or a severity level thereof may be provided by a patient and/or a medical professional, and/or substitution system 102 may analyze medical claims and/or Electronic Health Records (EHR) associated with the patient to determine a confirmed diagnosis and/or a severity level thereof associated with the patient.

As shown in FIG. 4, at step 412, process 400 includes, in response to determining that the patient identifier associated with the unique hash (and/or the upgraded hash(es)) is mapped to a confirmed diagnosis, generating a confirmed hash. For example, substitution system 102 may, in response to determining that the patient identifier associated with the unique hash LikelyHASH (and/or the upgraded hash(es) UpgradedHASH(es)) is mapped to a confirmed diagnosis, generate a confirmed hash ConfirmedHASH(CUI1, CUI2, . . . CIUn). As an example, substitution system 102 may combine the patient identifier associated with the unique hash LikelyHASH (and/or the upgraded hash(es) UpgradedHASH(es)) with the confirmed diagnosis associated with the same patient identifier to generate the confirmed hash ConfirmedHASH(CUI1, CUI2, . . . CIUn).

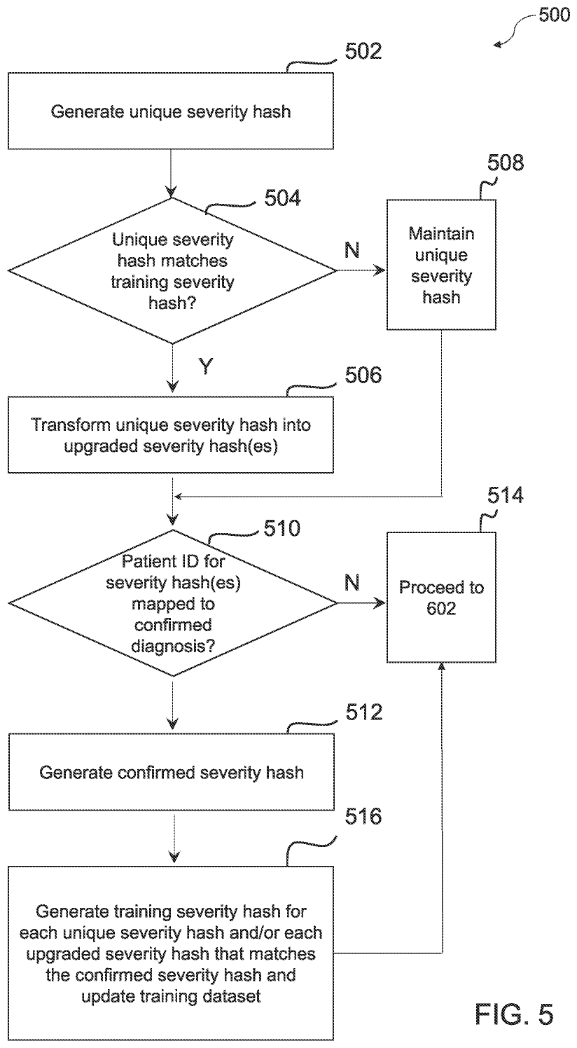

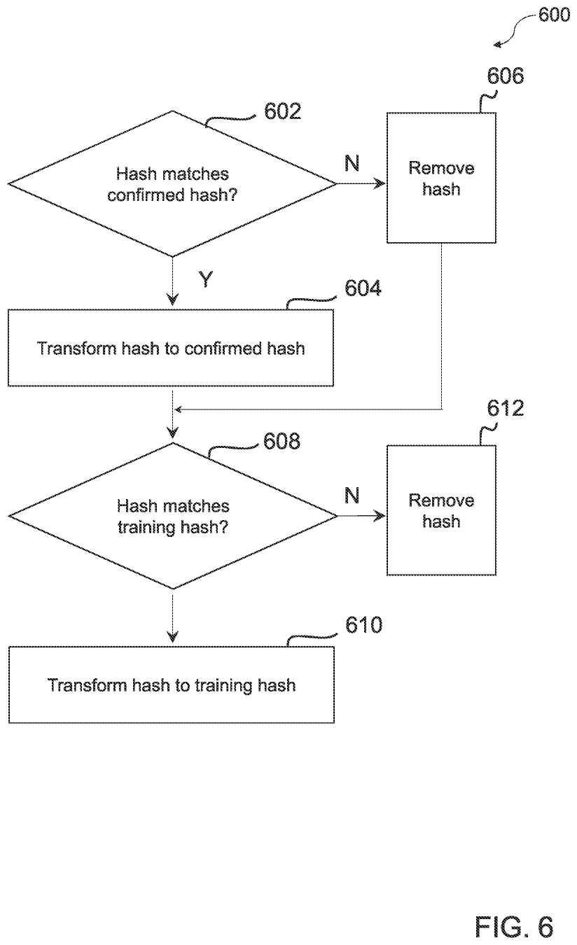

As shown in FIG. 4, at step 414, process 400 includes, in response to determining that the patient identifier associated with the unique hash (and/or the upgraded hash(es)) is not mapped to a confirmed diagnosis, processing may proceed directly to step 502 of process 500 in FIG. 5 or directly to step 602 of process 600 in FIG. 6. For example, substitution system 102 may, in response to determining that the patient identifier associated with the unique hash LikelyHASH (and/or the upgraded hash(es) UpgradedHASH(es)) is not mapped to a confirmed diagnosis, proceed to step 418 without generating a confirmed hash. In such an example, an upgraded hash UpgradedHASH may be associated with a greater probability of diagnosis than a unique hash LikelyHASH that has not been upgraded/transformed. As an example, substitution system 102 may provide a list of the upgraded hash(es) UpgradedHASH(es) and the unique hash LikelyHASH.

As shown in FIG. 4, at step 416, process 400 includes, in response to generating a confirmed hash, generating a training hash for each unique hash (and/or each upgraded hash(es)) that matches the confirmed hash and updating the training dataset based on the generated training hash. For example, substitution system 102 may, in response to generating a confirmed hash ConfirmedHASH, generate a training hash TrainingHASH for each unique hash LikelyHash and/or each upgraded hash(es) UpgradedHASH(es) that matches the confirmed hash ConfirmedHASH and update the training dataset based on the generated training hash TrainingHASH. As an example, if a same (e.g., a matching or equivalent hash, etc.) training hash as the generated training hash TrainingHASH is not already stored in the training dataset, substitution system 102 may update the training dataset to include the generated training hash TrainingHASH, and if the same training hash as the generated training hash TrainingHASH is already stored in the training dataset, substitution system 102 may update the training dataset by adjusting a weighting associated with the training hash TrainingHASH (e.g., increment a count associated with the training hash TrainingHASHby one in the training dataset, etc.). In such an example, substitution system 102 may provide a list of the confirmed hash(es) Con firmedHASH(es), the upgraded hash(es) UpgradedHASH(es), and the unique hash LikelyHASH in descending order of likelihood that a hash represents the prescribed drug(s) a patient is taking for each diagnosis.

Accordingly, after sufficient iterations of process 400, the training hash(es) TrainingHASH(es) for each diagnosis in the training dataset may become stronger and/or more statistically likely to be correct diagnoses, which may be reflected by the upgraded hash UpgradedHASH used in step 406. For example, each action taken by a clinician may be turned into incremental knowledge that is adjusted overtime and, if multiple clinicians begin to dismiss or ignore potential substitutions for a particular prescription for a particular type of patient (e.g., men ages 30-40, etc.), substitution system 102 may learn to consider the patient attributes associated with that particular type of patient.

Referring now also to FIG. 5, FIG. 5 is a flowchart of non-limiting embodiments or aspects of a process 500 for pharmacy substitutions. In some non-limiting embodiments or aspects, one or more of the steps of process 500 may be performed (e.g., completely, partially, etc.) by substitution system 102 (e.g., one or more devices of substitution system 102, etc.). In some non-limiting embodiments or aspects, one or more of the steps of process 500 may be performed (e.g., completely, partially, etc.) by another device or a group of devices separate from or including substitution system 102, such as a user device (e.g., one or more devices of a system of a user device, etc.).

As shown in FIG. 5, at step 502, process 500 includes generating a unique severity hash associated with a patient. For example, substitution system 102 may generate a unique severity hash LikelySeverityHASH associated with a patient by combining a patient identifier associated with a universal identifier (e.g., as generated at step 304, etc.) with severity of a diagnosis associated with the same universal identifier (e.g., the same RxNorm CUI, etc.). As an example, the unique severity hash LikelySeverityHASH may include an ascending list of universal identifiers (e.g., CUls, etc.) for each likely severity of each diagnosis (e.g., mild diabetes, moderate diabetes, severe diabetes, etc.) for each patient.

A patient may be associated with a plurality of unique severity hashes LikelySeverityHASHes if the patient is associated with multiple diagnoses. For example, for two example patients Patient 1 and Patient 2, the following example unique severity hashes may be generated: Patient 1: LikelySeverityiHASH(CUI1,CUI2,CUI3) equals likely severe Diabetes for Patient 1, Patient 2: LikelySeverityHASH(CU1,CUI2,CUI5) equals likely moderate Diabetes for Patient 2, and Patient 1: LIkelySeverityHASH(CUI4,CUI5) equals likely severe Ketoacidosis for Patient 1. As an example, a unique severity hash LikelySeverityHASH may include any number of CUls for a particular severity of a diagnosis, and/or CUls may overlap for different severities diagnoses.

As shown in FIG. 5, at step 504, process 500 includes determining whether a unique severity hash matches a training severity hash. For example, substitution system 102 may determine whether the unique severity hash LikelySeverityHASH matches a TrainingSeverityHASH stored in a training dataset. As an example, substitution system 102 may compare each unique severity hash LikelySeverityHASH to TrainingSeverityHASHes previously stored in a training dataset.

As shown in FIG. 5, at step 506, process 500 includes, in response to determining that a unique severity hash matches a training severity hash, transforming the unique severity hash into one or more upgraded severity hashes. For example, substitution system 102 may, in response to determining that the unique severity hash LikelySeverityHASH matches one or more TrainingSeverityHASHes stored in the training dataset, transform the unique severity hash LikelySeverityHASH into one or more upgraded severity hash(es) UpgradedSeverityHASH(es). As an example, for each match that the unique severity hash LikelySeverityHASH has in the training dataset, an upgraded severity hash UpgradedSeverityHASH may be generated such that a single unique severity hash LikelySeverityHASH may generate multiple UpgradedSeverityHASHes.

As shown in FIG. 5, at step 508, process 500 includes, in response to determining that a unique severity hash does not match a training severity hash, maintaining the unique severity hash. For example, substitution system 102 may, in response to determining that the unique severity hash LikelySeverityHASH does not match any TrainingSeverityHASH stored in the training dataset, maintain the unique severity hash LikelySeverityHASH (e.g., not transform the unique severity hash LikelySeverityHASH, etc.).

As shown in FIG. 5, at step 510, process 500 includes determining whether a patient identifier associated with a unique severity hash (and/or an upgraded severity hash) is mapped to a confirmed severity of a diagnosis. For example, substitution system 102 may determine whether a patient identifier associated with the unique severity hash identifier LikelySeverityHASH (and/or the upgraded severity hash(es) UpgradedSeverityHASH(es)) is mapped to a confirmed severity of a diagnosis. As an example, substitution system 102 may use a mapping that maps patient identifiers to confirmed severities of diagnoses for the patients associated with the patient identifiers. For example, a confirmed severity of a diagnosis may be provided by a patient and/or a medical professional, and/or substitution system 102 may analyze medical claims associated with the patient to determine a confirmed severity of a diagnosis associated with the patient.

As shown in FIG. 5, at step 512, process 500 includes, in response to determining that the patient identifier associated with the unique severity hash (and/or the upgraded severity hash(es)) is mapped to a confirmed severity of a diagnosis, generating a confirmed severity hash. For example, substitution system 102 may, in response to determining that the patient identifier associated with the unique severity hash LikelySeverityHASH (and/or the upgraded severity hash (es) UpgradedSeverityHASH(es)) is mapped to a confirmed severity of a diagnosis, generate a confirmed severity hash ConfirmedSeverityHASH(CUI1, CUI2, . . . CIUn). As an example, substitution system 102 may combine the patient identifier associated with the unique severity hash LikelySeverityHASH (and/or the upgraded severity hash(es) UpgradedSeverityHASH(es)) with the confirmed severity of a diagnosis associated with the same patient identifier to generate the confirmed severity hash ConfirmedSeverityHASH(CUI1, CUI2, . . . CIUn).

As shown in FIG. 5, at step 514, process 500 includes, in response to determining that the patient identifier associated with the unique severity hash (and/or the upgraded severity hash(es)) is not mapped to a confirmed severity of a diagnosis, processing may proceed directly to step 602 of process 600 in FIG. 6. For example, substitution system 102 may, in response to determining that the patient identifier associated with the unique severity hash LikelySeverityHASH (and/or the upgraded severity hash(es) UpgradedSeverityHASH(es)) is not mapped to a confirmed severity of a diagnosis, proceed to step 518 without generating a confirmed severity hash. In such an example, an upgraded severity hash UpgradedSeverityHASH may be associated with a greater probability of a severity of a diagnosis than a unique severity hash LikelySeverityHASH that has not been upgraded/transformed. As an example, substitution system 102 may provide a list of the upgraded severity hash(es) UpgradedSeverityHASH(es) and the unique severity hash LikelySeverityHASH.

As shown in FIG. 5, at step 516, process 500 includes, in response to generating a confirmed severity hash, generating a training severity hash for each unique severity hash (and/or each upgraded severity hash(es)) that matches the confirmed severity hash and updating the training dataset based on the generated training severity hash. For example, substitution system 102 may, in response to generating a confirmed severity hash Con firmedSeverityHASH, generate a training severity hash TrainingSeverityHASH for each unique severity hash LikelySeverityHash and/or each upgraded severity hash(es) UpgradedSeverityHASH(es) that matches the confirmed severity hash ConfirmedHASH and update the training dataset based on the generated training hash TrainingSeverityHASH. As an example, if a same (e.g., a matching or equivalent hash, etc.) training severity hash as the generated training severity hash TrainingSeverityHASH is not already stored in the training dataset, substitution system 102 may update the training dataset to include the generated training severity hash TrainingSeverityHASH, and if the same training severity hash as the generated training severity hash TrainingSeverityHASH is already stored in the training dataset, substitution system 102 may update the training dataset by adjusting a weighting associated with the training severity hash TrainingSeverityHASH (e.g., increment a count associated with the training severity hash TrainingSeverityHASH by one in the training dataset, etc.). In such an example, substitution system 102 may provide a list of the confirmed severity hash(es) Con firmedSeverityHASH(es), the upgraded severity hash (es) UpgradedSeverityHASH(es), and the unique severity hash LikelySeverityHASH in descending order of likelihood that a severity hash represents the prescribed drug(s) a patient is taking for each diagnosis and the severity of that diagnosis.

Accordingly, after sufficient iterations of process 500, the training severity hash(es) TrainingSeverityHASH(es) for each severity of each diagnosis in the training dataset may become stronger and/or more statistically likely to be correct severities of diagnoses, which may be reflected by the upgraded severity hash UpgradedSeverityHASH used in step 506. For example, considering a severity of a diagnosis may enable clinically equivalent prescriptions may be determined based on the severity of a unique medical situation of a patient. As an example, each patient with moderate Crohn's disease may receive drug ABC, and/or patients with severe Crohn's disease may receive drugs DEF+XYZ.

Example of Clinical Equivalency based on Severity of Diagnosis

For an example patient with a diagnosis of Rheumatoid Arthritis, if substitution system 102 does not consider or know severity, severity system 102 may determine that it is clinically equivalent to substitute Ibuprofen for Humira for cost purposes, which may result in the following output/substitutions: Highest Cost: Humira 40 mg-$100/month; Lower Cost Option 1: Enbrel 50 mg-$75/month; Lower Cost Option 2: Methotrexate 2.5 mg and Hydroxychloroquine Sulfate 200 mg-$50/month; Lower Cost Option 3: Methotrexate 2.5 mg-$25/month; and Lower Cost Option 4: Ibuprofen 800 mg-$10/month. However, when substitution system 102 knows and considers severity, the output/substitutions may change because substitution system 102 may learn that Ibuprofen is not clinically equivalent for a severe diagnosis of Rheumatoid Arthritis, which may result in the following output/substitutions: Highest Cost: Humira 40 mg-$100/month; Lower Cost Option 1: Enbrel 50 mg-$75/month; Lower Cost Option 2: Methotrexate 2.5 mg and Hydroxychloroquine Sulfate 200 mg-$50/month.

Referring now also to FIG. 6, FIG. 6 is a flowchart of non-limiting embodiments or aspects of a process 600 for pharmacy substitutions. In some non-limiting embodiments or aspects, one or more of the steps of process 600 may be performed (e.g., completely, partially, etc.) by substitution system 102 (e.g., one or more devices of substitution system 102, etc.). In some non-limiting embodiments or aspects, one or more of the steps of process 600 may be performed (e.g., completely, partially, etc.) by another device or a group of devices separate from or including substitution system 102, such as a user device (e.g., one or more devices of a system of a user device, etc.).

As shown in FIG. 6, at step 602, process 600 includes determining whether a hash matches a confirmed hash. For example, substitution system 102 may process the list of the confirmed hash(es) Con firmedHASH(es), the upgraded hash(es) UpgradedHASH(es), and the unique hash LikelyHASH and/or the list of the confirmed severity hash(es) Con firmedSeverityHASH(es), the upgraded severity hash(es) UpgradedSeverityHASH(es), and the unique severity hash LikelySeverityHASH to determine whether each hash and/or severity hash matches confirmed hash/severity hash. As an example, substitution system 102 may perform step 602 to clean-up the hash data by removing known bad hashes (e.g., non-equivalent drug to diagnosis hashes, hashes without a matching diagnosis, etc.) if confirmed patient to diagnosis data is available. If confirmed patient to diagnosis data is not available, substitution system 102 may proceed directly to step 608.

As shown in FIG. 6, at step 604, process 600 includes, in response to determining that a hash matches a confirmed hash, transforming the hash into a confirmed hash. For example, substitution system 102 may transform the upgraded hash(es) UpgradedHASH(es)/the unique hash LikelyHASH and/or the upgraded severity hash(es) UpgradedSeverityHASH(es)/the unique severity hash LikelySeverityHASH into the confirmed hash(es) ConfirmedHASH(es) and/or Con firmedSeverityHASH(es) in response to the upgraded hash(es) UpgradedHASH(es)/the unique hash LikelyHASH and/or the upgraded severity hash(es) UpgradedSeverityHASH(es)/the unique severity hash LikelySeverityHASH matching a confirmed hash ConfirmedHASH(es) and/or ConfirmedSeverityHASH(es).

As shown in FIG. 6, at step 606, process 600 includes, in response to determining that a hash does not match a confirmed hash, removing or deleting the hash. For example, substitution system 102 may remove or delete the upgraded hash(es) UpgradedHASH(es)/the unique hash LikelyHASH and/or the upgraded severity hash(es) UpgradedSeverityHASH(es)/the unique severity hash LikelySeverityHASH in response to the upgraded hash(es) UpgradedHASH(es)/the unique hash LikelyHASH and/or the upgraded severity hash(es) UpgradedSeverityHASH(es)/the unique severity hash LikelySeverityHASH not matching a confirmed hash ConfirmedHASH(es) and/or ConfirmedSeverityHASH(es).

As shown in FIG. 6, at step 608, process 600 includes determining whether a hash matches a training hash. For example, substitution system 102 may process the list of the confirmed hash(es) Con firmedHASH(es), the upgraded hash(es) UpgradedHASH(es), and the unique hash LikelyHASH and/or the list of the confirmed severity hash(es) Con firmedSeverityHASH(es), the upgraded severity hash(es) UpgradedSeverityHASH(es), and the unique severity hash LikelySeverityHASH to determine whether each hash and/or severity hash matches a training hash/training severity hash. As an example, substitution system 102 may use the training dataset to remove hash(es) where there is no diagnosis match (based upon enough prescribing physicians refusing to make the substitution (e.g., discovered input, etc.), and/or pharmacists (e.g., keyed-in input, etc.) removing the drug to diagnosis match.

As shown in FIG. 6, at step 610, process 600 includes, in response to determining that a hash matches a training hash, transforming the hash into a training hash. For example, substitution system 102 may transform the upgraded hash(es) UpgradedHASH(es)/the unique hash LikelyHASH and/or the upgraded severity hash(es) UpgradedSeverityHASH(es)/the unique severity hash LikelySeverityHASH into the training hash(es) TrainingHASH(es) and/or TrainingSeverityHASH(es) in response to the upgraded hash(es) UpgradedHASH(es)/the unique hash LikelyHASH and/or the upgraded severity hash(es) UpgradedSeverityHASH(es)/the unique severity hash LikelySeverityHASH matching a training hash TrainingHASH(es) and/or TrainingSeverityHASH(es).

As shown in FIG. 6, at step 612, process 600 includes, in response to determining that a hash does not match a training hash, removing or deleting the hash. For example, substitution system 102 may remove or delete the upgraded hash(es) UpgradedHASH(es)/the unique hash LikelyHASH and/or the upgraded severity hash(es) UpgradedSeverityHASH(es)/the unique severity hash LikelySeverityHASH in response to the upgraded hash(es) UpgradedHASH(es)/the unique hash LikelyHASH and/or the upgraded severity hash(es) UpgradedSeverityHASH(es)/the unique severity hash LikelySeverityHASH not matching a training hash TrainingHASH(es) and/or TrainingSeverityHASH(es).

Accordingly, an output of substitution system 102 from process 600 may include the hashes that have been discovered as more likely to reflect a drug to diagnosis equivalency or, if patient to diagnosis data is not available for step 602, newly found hashes for which substitution system 102 does not have previous training for that drug to diagnosis equivalency.

Referring again to FIG. 3A, at step 310, process 300 includes determining a cost associated with a diagnosis. For example, substitution system 102 may determine, based on the claims data, at least one cost associated with the at least one likely diagnosis associated with the at least one prescription. As an example, substitution system 102 may determine the at least one cost based on a current cost associated with the at least one prescription and a future cost associated with the at least one prescription, wherein the future cost is different than the current cost. In such an example, substitution system 102 may determine a daily patient cost of an entire hash associated with a patient by taking a sum of an insurance and/or other drug benefit payor amount paid and/or a patient copay and/or other payment arrangement (e.g. flat percentage, drug deductible limit, etc.) amount paid for each drug in the hash and dividing the sum by a number of days between a minimum date filled and a maximum next fill date. For example, an output of substitution system 102 from step 310 may include the patient identifier, the hashes (e.g., (ConfirmedHASH/ConfirmedSeverityHASH, UpgradedHASH/UpgradedSeverityHASH, and/or LikelyHASH/LikelySeverityHASH), and the determined daily cost.

A cost associated with a diagnosis and/or a prescription may include at least one of the following costs: a current financial cost (e.g., in dollars, euros, etc.), a time cost (e.g., an amount of time to take/receive a drug, etc.), a predicted outcome to cost ratio, a side effect cost (e.g., a severity level of side effects associated with a drug, etc.), a future financial cost (e.g., paying somewhat more financially initially to avoid a predicted larger financial cost in the future, for example, it may be financially advantageous to change prescriptions to pay X+5 initially to avoid paying a likely X+100 cost in the future, etc.), a patient satisfaction cost, or any combination thereof.

In some non-limiting embodiments or aspects, substitution system 102 uses a future dataset including future data associated with future drug prices (e.g., known future negotiated drug pricing provided by an insurer or other payor of drug benefits, publicly disclosed drug manufacturer pricing increases, etc.) and future clinically equivalent drugs arriving on the market (e.g., a generic alternative to an existing drug is announced, etc.). For example, future data may include at least one of the following parameters: a drug name, a future price associated with the drug, a future effective/available date associated with the drug, a diagnosis or equivalent drug to the drug, or any combination thereof.

As shown in FIG. 3B, at step 312, process 300 includes determining a predicted total cost. For example, substitution system 102 may determine, for a patient, a predicted total cost per-day cost, per diagnosis, for each of the drugs combined in each hash associated with the patient identifier of the patient. As an example, substitution system 102 may determine an expected future daily patient cost of the entire hash by taking the sum of the anticipated/expected insurance amount to be paid and/or the patient copay and/or other payment arrangement amount paid for each drugs in the hash and dividing the sum by a typical number of days between a minimum date filled and a maximum next fill date. For example, an output of substitution system 102 from step 312 may include the patient identifier, the hashes (e.g., ConfirmedFutureHASH/ConfirmedFutureSeverityHASH, UpgradedFutureHASH/UpgradedFutureSeverityHASH, and/or LikelyFutureHASH/LikelyFutureSeverityHASH), and the predicted total cost.

As shown in FIG. 3B, at step 314, process 300 includes determining a total historic and future cost. For example, substitution system 102 may determine, for the patient, the total historic (from step 310) and future (from 312) cost of a drug regimen for a given diagnosis for an organization (e.g., an insurer, a payor of drug benefits, a cohort, a company, an accountable care organization, etc.) and the individual Lines of Business of the organization (e.g., Gold Plan, Silver Plan, Public or Private coverage, etc.). As an example, substitution system 102 may, for each unique group hash, count a total number of patients associated with the hash and the total amount of daily cost for the hash. In such an example, substitution system 102 may store the common prescribed drugs available in a given Line of Business or national level formulary, future formulary details, and/or or upcoming drug release details from the insurer or other payor of drug benefits for each Line of Business or drug benefit covered group. For example, substitution system 102 may receive and/or determine which drugs and dosages, from all possible drugs, can be used for substitution purposes for a given patient to avoid suggesting a substitution where the drug isn't available to the patient because of Line of Business contract restrictions or pharmacy benefits management, which can lead to false learning cycles. In such an example, an output of substitution system 102 from step 314 may include the hash, the number of patients associated with the hash, and/or the daily cost.

As shown in FIG. 3B, at step 316, process 300 includes determining that the group hash includes a threshold number of patients. For example, substitution system 102 may determine that the group hash (e.g., the at least one likely diagnosis associated with the at least one prescription, etc.) includes at least a threshold number of patients. As an example, substitution system 102 may determine the threshold number of patients based on a percentage of a total number of patients analyzed by substitution system 102 and considering a statistical likelihood of the diagnosis for a given patient population. In such an example, an output of substitution system 102 from step 314 may include the hash, the number of patients, and the daily cost. It is noted that in step 314, if the threshold number of patients is not met, the hashes may be added to the training dataset for future consideration without being output for current use in step 318.

As shown in FIG. 3B, at step 318, process 300 includes determining at least one potential alternative prescription to at least one prescription. For example, substitution system 102 may determine, for the at least one likely diagnosis, using a machine learning model trained based on the training dataset, at least one potential alternative prescription to the at least one prescription associated with the at least one likely diagnosis. As an example, substitution system 102 may generate potential rules for drug(s) substitution for prescriptions where replacing a higher cost hash with a more economical lower cost hash results in a similar clinical effectiveness for a similar severity of diagnosis. In such an example, substation system 102 may use at least one of the following conditions to generate a drug substitution for a drug(s): a clinically equivalent drug(s) exists, a lower cost alternative drug(s) or drug(s) dosages have been determined by substitution system 102, a likely diagnosis(es) supports substitution for optional severity of diagnosis, an anticipated cost of care can be decreased (and/or quality of care increased) by a patient beginning a prescription for a lower cost drug for a new diagnosis, or any combination thereof. For example, substitution system 102 may generate a list of one (or multiple) groups of drugs, based upon descending likelihood of hash (Confirmed, Upgraded, Likely), that can be substituted for one (or multiple) alternative drugs, for a given diagnosis and optional severity, and/or substitution system 102 may order or rank these results based on each of a likelihood and an overall economic savings, where the savings meets a pre-calculated minimum threshold.

Substitution system 102 may generate the machine learning model (e.g., an estimator, a classifier, a prediction model, a detector model, etc.) using machine learning techniques including, for example, supervised and/or unsupervised techniques, such as decision trees (e.g., gradient boosted decision trees, random forests, etc.), logistic regressions, artificial neural networks (e.g., convolutional neural networks, etc.), Bayesian statistics, learning automata, Hidden Markov Modeling, linear classifiers, quadratic classifiers, association rule learning, and/or the like. The machine learning model may be trained to provide an output including at least one alternative prescription (e.g., a list of one (or multiple) groups of drugs, based upon descending likelihood of hash (Confirmed, Upgraded, Likely), etc.) in response to input including at least one prescription associated with at least one diagnosis (e.g., at least one prescription associated with a given diagnosis and optional severity, the group hash, etc.). For example, substitution system 102 may train the model based on training data (e.g., the training dataset, training hashes, etc.) associated with one or more prescriptions for one or more diagnoses for one or more patients. In such an example, an alternative prescription may include a probability score associated with the alternative prescription. For example, the alternative prescription may include a probability that the alternative prescription is a more economical lower cost drug(s) that results in a similar clinical effectiveness for a similar diagnosis/severity of diagnosis.

In some non-limiting embodiments, substitution system 102 may store the model (e.g., store the model for later use). In some non-limiting embodiments or aspects, substitution system 102 may store the model in a data structure (e.g., a database, a linked list, a tree, etc.). In some non-limiting embodiments, the data structure is located within substitution system 102 or external (e.g., remote from) substitution system 102 (e.g., within a data source 104, etc.).

A potential alternative prescription (or potential discovered rule) may include at least one of the following: a different drug than a drug associated with the at least one prescription (e.g., a single drug for a single drug substitution, a single drug for multiple drugs substitution, a multiple drug for a single drug substitution, etc.) , a different dosage than a dosage associated with the at least one prescription, an indication to discontinue use of the drug associated with the at least one prescription, a different formulation of the same drug associated with the at least one prescription (e.g., a tablet vs. a capsule, an oral delivery vs. an injection delivery, etc.), a different packaging of the same drug associated with the at least one prescription (e.g., over-the-counter, generic, brand-name, etc.), or any combination thereof.

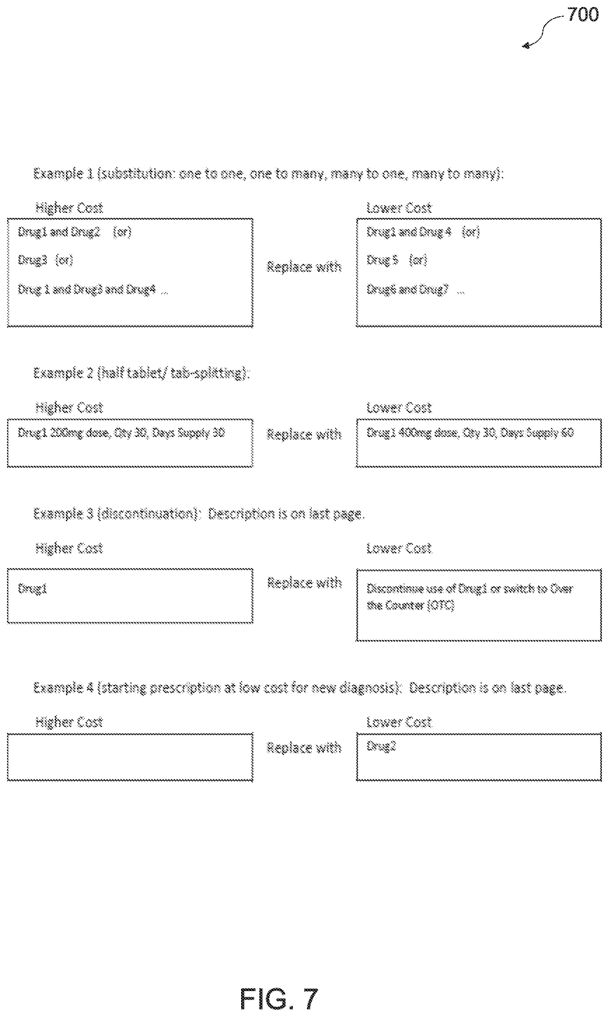

Referring also to FIG. 7, FIG. 7 illustrates example potential rules 700 that may be discovered by process 300. These rules may also consider and/or suggest an age or gender of the patient taking the prescription, other additional diagnoses of the patient, expected duration of each of the higher and lower cost drug regimen, clinical safety and quality of care (e.g., side-effects, etc.) for the drug regimen, and/or limitations on the available drugs in a Line of Business's or at national level available drug formulary.

Referring specifically to Example 3 in FIG. 7, substitution system may substitute a drug(s) for a lack of a drug(s). For example, substitution system 102 may use the following criteria to substitute a drug(s) for a lack of a drug(s): the patient is on multiple prescriptions, which enables determining that the patient is alive and still a member of the insurer or another coverage group (e.g., citizenship, etc.) of drug benefits, the patient has not switched to another drug(s) for the same diagnosis, substitution system 102 has not received new claims data for a predetermined period after the next fill date, and a predetermined threshold is satisfied where the system has recognized that enough patients have “discontinued”, enabling substitution system 102 to identify that a potential discovered rule for a discontinuation may be possible.

Referring specifically to Example 4 in FIG. 7, substitution system may start a prescription at a low cost for a new diagnosis. For example, substitution system 102 may train prescribers on cost savings. As an example, if two drugs are used to treat the same diagnosis, where one drug is more costly than the other, substitution system 102 may identify when a prescriber commonly begins new prescriptions, for a newly diagnosed patient, with the lower cost drug, and substitution system 102 may consider the prescriber to be “trained” for the potential discovered rule. Identification of prescriber habits enables identifying opportunities to educate the prescriber on better/cost saving habits. Starting prescriptions at the low cost for a new diagnosis may also result in a positive reinforcement (e.g., an increment of +1, etc.) of the corresponding hashes in the training dataset. In such an example, substitution system 102 may use the following criteria to start a prescription at a low cost for a new diagnosis: a prescriber is prescribing a higher cost drug(s) for a given diagnosis, substitution system 102 identifies a new patient diagnosis, the prescriber begins prescribing the lower cost drug(s) for the new patient diagnosis, and a threshold of new prescriptions of the lower cost drug(s) for the new patient diagnosis is satisfied. Similarly, there are occasions where beginning a prescription for a low-cost drug immediately (for a given diagnosis), may prevent or delay the need for a very high cost drug at a later time because of increase in diagnosis severity. Identifying these patients and suggesting a high-cost substitution of “nothing” to be replaced by the low-cost drug decreases the overall cost of care and possibly increases the quality of care. Example 4 may also be used to accomplish this scenario, for example, for example, and as previously described herein, it may be financially advantageous to change prescriptions to pay X+5 initially to avoid paying a likely X+100 cost in the future, etc.).

Accordingly, when comparing potential rules generated by non-limiting embodiments or aspects of the present disclosure to manually generated potential rules created by Pharmacist's/PharmD's, non-limiting embodiments or aspects of the present disclosure may independently discover 90% of the rules that Pharmacists may otherwise discover and independently discover 254% more potential rules and drug substitutions for savings that Pharmacist's may not otherwise discover.

As shown in FIG. 3B, at step 320, process 300 includes providing savings information. For example, substitution system 102 may provide, to at least one user (e.g., a prescriber, etc.), savings information associated with the at least one potential alternative prescription to the at least one prescription associated with the at least one likely diagnosis. As an example, substitution system 102 may determine an opportunity for a cost savings difference between a higher cost drug(s) and a lower cost drug(s), multiplied by a factor (e.g., 1%, 5%, etc.), to determine if a copay or other payment arrangement (e.g. flat percentage, drug deductible limit, etc.) of a patient may limit the likelihood of the patient agreeing to the lower cost alternative (e.g., to determine if the patient's portion of cost (if any) may limit the likelihood of the patient agreeing to the substitution, etc.), and if substitution system determines that the copay or other payment arrangement may limit the willingness of the patient to substitute drug(s), flag the opportunity for the cost savings as a potential for copay or other payment arrangement reimbursement and follow a process for offering payment by sending a message to patient about the opportunity and/or to discuss with prescriber. In such an example, an output of substitution system 102 from step 320 may include the opportunity for cost savings, per patient, with a flag indicating whether the copay or other payment arrangement may limit the willingness of the patient to substitute drug(s). For example, substitution system 102 may display the opportunity for savings to prescriber in an application and/or send message (e.g., an email, a SMS message, etc.) to the prescriber including the opportunity for savings.

An opportunity for savings may include at least one of the following parameters: patient name, patient date of birth, patient gender, high cost drug(s), a number of days of supply of drug(s), a last filled date of a drug(s), a next fill expected date of drug(s), a prescriber name, an insurer or other payor of drug benefits paid amount, a patient paid amount, lower cost drug(s), a date the opportunity for savings was discovered, a potential savings for making a substitution, or any combination thereof. In some non-limiting embodiments or aspects, an opportunity for savings may include one or more of the following meta-details: a current status of the opportunity for savings (e.g., Ready, Completed, Dismissed—Patient Refuses, Dismissed—Patient did not tolerate, Dismiss—Opportunity Inaccurate, etc.), a history and feedback of the opportunity for savings, notes about the drug(s) included, or any combination thereof.

As shown in FIG. 3B, at step 322, process 300 includes receiving user input associated with at least one potential alternative prescription. For example, substitution system 102 may receive, from the at least one user (e.g., the prescriber, etc.), user input associated with the at least one potential alternative prescription to the at least one prescription associated with the at least one likely diagnosis. As an example, a prescriber may verify whether or not they agree with the at least one potential alternative prescription (e.g., with a discovered Potential Rule(s), etc.).

As shown in FIG. 3B, at step 324, process 300 includes updating a training dataset. For example, substitution system 102 may update the training dataset to include the at least one potential alternative prescription (e.g., the discovered Potential Rule(s), etc.). As an example, substitution system 102 may update the training dataset to include the at least one potential alternative prescription (e.g., the discovered Potential Rule(s), etc.) in response to a number of prescribers verifying that they do not agree with the at least one potential alternative prescription (e.g., the discovered Potential Rule(s), etc.) failing to satisfy a threshold number. In such an example, if a high percentage (e.g., a percentage satisfying a threshold percentage, etc.) of prescribers verify that they do not agree with the at least one potential alternative prescription (e.g., with the discovered potential rule(s), with the substitution, etc.), substitution system 102 may update the training dataset to remove a possibility for this substitution or alternative prescription to be recommended, and if a low percentage (e.g., a percentage failing to satisfy a threshold percentage, etc.) of prescribers verify that they do not agree with the at least one potential alternative prescription (and/or verify that they do agree with the at least one potential alternative prescription) substitution system 102 may update the training dataset to remove a possibility for this substitution or alternative prescription to be recommended.

Substitution system 102 may determine whether to update the training dataset using the list of the confirmed hash(es) Con firmedHASH(es), the upgraded hash(es) UpgradedHASH(es), and the unique hash LikelyHASH and/or the list of the confirmed severity hash(es) Con firmedSeverityHASH(es), the upgraded severity hash(es) UpgradedSeverityHASH(es), and the unique severity hash LikelySeverityHASH (e.g., using the outputs of steps 416 and/or 516, using automatic training, et.), using the updated training dataset updated based on the user input (e.g.,. trained by prescribers, etc.) and/or using a training process as described herein below in more detail (e.g., training by pharmacists or other authoritative source, etc.). A highest “credibility” or weight (e.g., probability, etc.) may be given to pharmacist decisions and a relatively high “credibility” or weight (e.g., probability, etc.) may be given to prescriber decisions when compared to the automatically discovered substitutions or rules (e.g., the outputs of steps 416 and/or 516, etc.), which may effectively provide a system where votes may be cast in favor or against a given potential discovered rule and the associated hash(es). For example, substation system 102 may determine, based on the at least one likely diagnosis and the training dataset, at least one probability associated with the at least one likely diagnosis, and the at least one probability may be input to the at least one machine learning model to determine the at least one potential alternative prescription to the at least one prescription associated with the at least one likely diagnosis.

Learned thresholds may determine the amount of “credibility” or weight votes required for a hash to no longer be considered for a diagnosis and associated alternative prescriptions/potential discovered rules, or accepted. Acceptance at a given time may not mean acceptance forever, as substitution system 102 may continuously learn and discover additional hashes and potential discovered rules. For example, if twenty prescribers accept an opportunity for cost savings (e.g., provide user input verifying the at least one potential alternative prescription to the at least one prescription associated with the at least one likely diagnosis, etc.) (and thereby the associated potential discovered rules and hashes) and make the substitution for the patient, it may cause substitution system 102 to “override” a single pharmacist rejecting the discovered rule for clinical efficacy reasons. Hashes may be sent back to the training dataset to impact future potential discovered rules. In such an example, an output of substitution system 102 at step 322 may include a final hash FinalHASH(CUI1,CUI2, . . . ) with diagnosis and optional severity of diagnosis.

As shown in FIG. 3B, at step 326, process 300 includes training a machine learning model based on an updated training dataset. For example, substitution system 102 may train (e.g., retrain, update, etc.) the machine learning model based on the updated training dataset. As an example, the machine learning model may be trained to provide an output including at least one alternative prescription (e.g., a list of one (or multiple) groups of drugs, based upon descending likelihood of hash (Confirmed, Upgraded, Likely), a potential discovered rule, etc.) in response to input including the updated training dataset (e.g., the training dataset updated to include at least one prescription associated with at least one diagnosis/the final hash, etc.). For example, substitution system 102 may train the model based on the updated training data (e.g., the updated training dataset, training hashes, the final hashes, etc.). In such an example, an alternative prescription or potential discovered rule may include a probability score associated with the alternative prescription. For example, the alternative prescription may include a probability that the alternative prescription is a more economical lower cost drug(s) that results in a similar clinical effectiveness for a similar diagnosis/severity of diagnosis. In such an example, substitution system 102 may store the updated model (e.g., store the updated model for later use. In such an example, substitution system 102 may continuously train and/or update the machine learning model as new claims data is received for new prescriptions for new and/or existing patients and/or as prescribers and/or pharmacists accept or reject proposed rules for alternative prescriptions.

Accordingly, providing (e.g., displaying, etc.) each alternative prescription or potential discovered rule with each associated diagnosis and with each associated hash (and the included drug(s)) with the ability for a pharmacist (and/or other authoritative source) to reject the potential discovered rule (and the associated hashes) for a given diagnosis may enable the pharmacist to reject rules based upon criteria, such as clinical efficacy, safety of the drug(s) or combination of the drug(s), concern for future cost (e.g., the pharmacist believes the lower cost drug will increase in price in the near future (where future anticipated cost data is not otherwise available), thereby invalidating the savings), etc.). A rejection reason that a pharmacist provides for a potential discovered rule may determine how substitution system 102 handles suggesting future potential discovered rules, and by a pharmacist not rejecting a potential discovered rule, substitution system 102 may assume implied acceptance of the potential discovered rule by that pharmacist. In this way, training may be based on any authoritative clinical interaction (e.g., from physicians, from pharmacists, from nurses, etc.) and/or from data analysis by substitution system 102.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, any of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. A computer-implemented method comprising:
obtaining claims data associated with at least one claim for at least one prescription associated with at least one patient;
determining, based on the claims data, at least one universal identifier for the at least one prescription associated with the at least one patient;
obtaining drug diagnosis data associated with one or more known diagnoses for one or more universal identifiers associated with one or more prescriptions;
determining, based on the at least one universal identifier and the drug diagnosis data, at least one likely diagnosis associated with the at least one prescription for the at least one patient;
determining, based on the claims data, at least one cost associated with the at least one likely diagnosis associated with the at least one prescription;
determining, for the at least one likely diagnosis, using a machine learning model trained based on a training dataset, at least one potential alternative prescription to the at least one prescription associated with the at least one likely diagnosis;
providing, to at least one user, savings information associated with the at least one potential alternative prescription to the at least one prescription associated with the at least one likely diagnosis;
receiving, from the at least one user, user input associated with the at least one potential alternative prescription to the at least one prescription associated with the at least one likely diagnosis;
updating, based on the user input, the training dataset to include the at least one likely diagnosis associated with the at least one potential alternative prescription as at least one trained diagnosis; and
training the machine learning model based on the updated training dataset.

2. The computer-implemented method of claim 1, further comprising:
determining whether the at least one likely diagnosis matches one or more confirmed diagnoses in a confirmed dataset;
in response to determining that the at least one likely diagnosis matches one or more confirmed diagnoses in a confirmed dataset, one of: (i) updating the training dataset to include the at least one likely diagnosis associated with the at least one prescription as one or more trained diagnosis and (ii) updating the trained dataset by adjusting a weighting associated with at least one existing trained diagnosis in the trained dataset.

3. The computer-implemented method of claim 2, further comprising:
determining, based on the at least one likely diagnosis and the confirmed data set, a severity level associated with the at least one likely diagnosis, wherein severity level is input to the at least one machine learning model to determine the at least one potential alternative prescription to the at least one prescription associated with the at least one likely diagnosis.

4. The computer-implemented method of claim 3, wherein the at least one potential alternative prescription is associated with a same severity level for the at least one likely diagnosis as the at least one prescription.

5. The computer-implemented method of claim 1, further comprising:
determining, based on the at least one likely diagnosis and the training dataset, at least one probability associated with the at least one likely diagnosis, wherein the at least one probability is input to the at least one machine learning model to determine the at least one potential alternative prescription to the at least one prescription associated with the at least one likely diagnosis.

6. The computer-implemented method of claim 1, wherein the at least one potential alternative prescription includes at least one of the following: a different drug than a drug associated with the at least one prescription, a different dosage than a dosage associated with the at least one prescription, an indication to discontinue use of the drug associated with the at least one prescription, a different formulation of the same drug associated with the at least one prescription, a different packaging of the same drug associated with the at least one prescription, or any combination thereof.

7. The computer-implemented method of claim 1, wherein the at least one cost is further determined based on a current cost associated with the at least one prescription and a future cost associated with the at least one prescription, wherein the future cost is different than the current cost.

8. A system comprising:
one or more processors programmed and/or configured to:
obtain claims data associated with at least one claim for at least one prescription associated with at least one patient;
determine, based on the claims data, at least one universal identifier for the at least one prescription associated with the at least one patient;
obtain drug diagnosis data associated with one or more known diagnoses for one or more universal identifiers associated with one or more prescriptions;
determine, based on the at least one universal identifier and the drug diagnosis data, at least one likely diagnosis associated with the at least one prescription for the at least one patient;
determine, based on the claims data, at least one cost associated with the at least one likely diagnosis associated with the at least one prescription;
determine, for the at least one likely diagnosis, using a machine learning model trained based on a training dataset, at least one potential alternative prescription to the at least one prescription associated with the at least one likely diagnosis;
provide, to at least one user, savings information associated with the at least one potential alternative prescription to the at least one prescription associated with the at least one likely diagnosis;
receive, from the at least one user, user input associated with the at least one potential alternative prescription to the at least one prescription associated with the at least one likely diagnosis;
update, based on the user input, the training dataset to include the at least one likely diagnosis associated with the at least one potential alternative prescription as at least one trained diagnosis; and
train the machine learning model based on the updated training dataset.

9. The system of claim 8, wherein the one or more processors are further programmed and/or configured to:
determine whether the at least one likely diagnosis matches one or more confirmed diagnoses in a confirmed dataset;
in response to determining that the at least one likely diagnosis matches one or more confirmed diagnoses in a confirmed dataset, one of: (i) update the trained dataset to include the at least one likely diagnosis associated with the at least one prescription as one or more trained diagnosis and (ii) update the trained dataset by adjusting a weighting associated with at least one existing trained diagnosis in the trained dataset.

10. The system of claim 9, wherein the one or more processors are further programmed and/or configured to:
determine, based on the at least one likely diagnosis and the confirmed data set, a severity level associated with the at least one likely diagnosis, wherein severity level is input to the at least one machine learning model to determine the at least one potential alternative prescription to the at least one prescription associated with the at least one likely diagnosis.

11. The system of claim 10, wherein the at least one potential alternative prescription is associated with a same severity level for the at least one likely diagnosis as the at least one prescription.

12. The system of claim 8, wherein the one or more processors are further programmed and/or configured to:
determine, based on the at least one likely diagnosis and the training dataset, at least one probability associated with the at least one likely diagnosis, wherein the at least one probability is input to the at least one machine learning model to determine the at least one potential alternative prescription to the at least one prescription associated with the at least one likely diagnosis.

13. The system of claim 8, wherein the at least one potential alternative prescription includes at least one of the following: a different drug than a drug associated with the at least one prescription, a different dosage than a dosage associated with the at least one prescription, an indication to discontinue use of the drug associated with the at least one prescription, a different formulation of the same drug associated with the at least one prescription, a different packaging of the same drug associated with the at least one prescription, or any combination thereof.

14. The system of claim 8, wherein the at least one cost is further determined based on a current cost associated with the at least one prescription and a future cost associated with the at least one prescription, wherein the future cost is different than the current cost.

15. A computer program product comprising at least one non-transitory computer-readable medium including program instructions that, when executed by at least one processor, cause the at least one processor to:
obtain claims data associated with at least one claim for at least one prescription associated with at least one patient;
determine, based on the claims data, at least one universal identifier for the at least one prescription associated with the at least one patient;
obtain drug diagnosis data associated with one or more known diagnoses for one or more universal identifiers associated with one or more prescriptions;
determine, based on the at least one universal identifier and the drug diagnosis data, at least one likely diagnosis associated with the at least one prescription for the at least one patient;
determine, based on the claims data, at least one cost associated with the at least one likely diagnosis associated with the at least one prescription;
determine, for the at least one likely diagnosis, using a machine learning model trained based on a training dataset, at least one potential alternative prescription to the at least one prescription associated with the at least one likely diagnosis;
provide, to at least one user, savings information associated with the at least one potential alternative prescription to the at least one prescription associated with the at least one likely diagnosis;
receive, from the at least one user, user input associated with the at least one potential alternative prescription to the at least one prescription associated with the at least one likely diagnosis;
update, based on the user input, the training dataset to include the at least one likely diagnosis associated with the at least one potential alternative prescription as at least one trained diagnosis; and
train the machine learning model based on the updated training dataset.

16. The computer program product of claim 15, wherein the instructions, when executed by the at least one processor, further cause the at least one processor to:
determine whether the at least one likely diagnosis matches one or more confirmed diagnoses in a confirmed dataset;
in response to determining that the at least one likely diagnosis matches one or more confirmed diagnoses in a confirmed dataset, one of: (i) update the trained dataset to include the at least one likely diagnosis associated with the at least one prescription as one or more trained diagnosis and (ii) update the trained dataset by adjusting a weighting associated with at least one existing trained diagnosis in the trained dataset.

17. The computer program product claim 16, wherein the instructions, when executed by the at least one processor, further cause the at least one processor to:
determine, based on the at least one likely diagnosis and the confirmed data set, a severity level associated with the at least one likely diagnosis, wherein severity level is input to the at least one machine learning model to determine the at least one potential alternative prescription to the at least one prescription associated with the at least one likely diagnosis, wherein the at least one potential alternative prescription is associated with a same severity level for the at least one likely diagnosis as the at least one prescription.

18. The computer program product of claim 15, wherein the instructions, when executed by the at least one processor, further cause the at least one processor to:
determine, based on the at least one likely diagnosis and the training dataset, at least one probability associated with the at least one likely diagnosis, wherein the at least one probability is input to the at least one machine learning model to determine the at least one potential alternative prescription to the at least one prescription associated with the at least one likely diagnosis.

19. The computer program product of claim 15, wherein the at least one potential alternative prescription includes at least one of the following: a different drug than a drug associated with the at least one prescription, a different dosage than a dosage associated with the at least one prescription, an indication to discontinue use of the drug associated with the at least one prescription, a different formulation of the same drug associated with the at least one prescription, a different packaging of the same drug associated with the at least one prescription, or any combination thereof.

20. The computer program product of claim 15, wherein the at least one cost is further determined based on a current cost associated with the at least one prescription and a future cost associated with the at least one prescription, wherein the future cost is different than the current cost.
